(19) United States
(12) Patent Application Publication
(10) Pub. No.: US 2010/0179434 A1
(43) Pub. Date: Jul. 15, 2010

(54) SYSTEMS AND METHODS FOR MAKING AND USING INTRAVASCULAR ULTRASOUND SYSTEMS WITH PHOTO-ACOUSTIC IMAGING CAPABILITIES

(75) Inventor: Peter Thornton, Los Altos, CA (US)

Correspondence Address: Boston Scientific Corporation;c/o Frommer Lawrence & Haug LLP, 745 Fifth Avenue, New York, NY, 10151, US

(73) Assignee: Boston Scientific SciMed, Inc., Maple Grove, MN (US)

(21) Appl. No.: 12/351,322

(22) Filed: Jan. 9, 2009

(51) Int. Cl. A61B 8/00; A61B 6/00

(52) U.S. Cl. 600/467; 600/476

(57) ABSTRACT

A catheter assembly for an intravascular ultrasound system includes a catheter and an imaging core disposed in the catheter. The imaging core includes a rotatable driveshaft, at least one light source, and at least one transducer. The at least one light source is disposed at a distal end of the rotatable driveshaft. The at least one light source is configured and arranged for rotating with the driveshaft and also for transforming applied electrical signals to light for illuminating an object in proximity to the catheter. The at least one transducer is also disposed at the distal end of the rotatable driveshaft. The at least one transducer is configured and arranged for rotating with the driveshaft. The at least one transducer is configured and arranged for receiving acoustic signals generated by the object in response to illumination of the object by the light emitted from the at least one light source.

### TECHNICAL FIELD

The present invention is directed to the area of intravascular ultrasound imaging systems and methods of making and using the systems. The present invention is also directed to intravascular ultrasound systems that also include photo-acoustic imaging, as well as methods of making and using the intravascular ultrasound systems.

### BACKGROUND

Intravascular ultrasound (“IVUS”) imaging systems have proven diagnostic capabilities for a variety of diseases and disorders. For example, IVUS imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as angiography and stent placement in real (or almost real) time. Moreover, IVUS imaging systems can be used to monitor one or more heart chambers.

IVUS imaging systems have been developed to provide a diagnostic tool for visualizing a variety is diseases or disorders. An IVUS imaging system can include a control module (with an electric pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The electric pulse generator in the control module generates electrical pulses that are delivered to the one or more transducers and transformed to acoustic pulses that are transmitted through patient tissue. Reflected pulses of the transmitted acoustic pulses are absorbed by the one or more transducers and transformed to electric pulses. The transformed electric pulses are delivered to the image processor and converted to an image displayable on the monitor.

Photo-acoustic imaging utilizes light and acoustic signals to form displayable images. In one exemplary photo-acoustic imaging technique, patient tissue is pulsed with light from a light source, such as a laser diode. Some of the emitted light is absorbed by the tissue and converted to heat. The heat causes a transient ultrasonic expansion of the illuminated tissue and a corresponding ultrasonic emission, which may be received by one or more transducers and processed into a displayable image.

### BRIEF SUMMARY

In one embodiment, a catheter assembly for an intravascular ultrasound system includes a catheter and an imaging core. The catheter has a distal end, a proximal end, and a longitudinal length, and defines a lumen extending along the longitudinal length of the catheter from the proximal end to the distal end. The imaging core is configured and arranged for inserting into the lumen. The imaging core includes a rotatable driveshaft, at least one light source, and at least one transducer. The rotatable driveshaft has a distal end, a proximal end, and a longitudinal length. The at least one light source is disposed at the distal end of the rotatable driveshaft. The at least one light source is configured and arranged for rotating with the driveshaft and also for transforming applied electrical signals to light for illuminating an object in proximity to the catheter. The at least one transducer is disposed at the distal end of the rotatable driveshaft. The at least one transducer is configured and arranged for rotating with the driveshaft. The at least one transducer is configured and arranged for receiving acoustic signals generated by the object in response to illumination of the object by the light emitted from the at least one light source.

In another embodiment, an intravascular ultrasound imaging system includes a catheter, an imaging core, and a drive unit. The catheter has a distal end, a proximal end, and a longitudinal length, and defines a lumen extending along the longitudinal length of the catheter from the proximal end to the distal end. The imaging core is configured and arranged for inserting into the lumen. The imaging core includes a rotatable driveshaft, at least one light source, and at least one transducer. The rotatable driveshaft has a distal end, a proximal end, and a longitudinal length. The at least one light source is disposed at the distal end of the rotatable driveshaft. The at least one light source is configured and arranged for rotating with the driveshaft and also for transforming applied electrical signals to light for illuminating an object in proximity to the catheter. The at least one transducer is disposed at the distal end of the rotatable driveshaft. The at least one transducer is configured and arranged for rotating with the driveshaft. The at least one transducer is configured and arranged for receiving acoustic signals generated by the object in response to illumination of the object by the light emitted from the at least one light source. The drive unit is coupled to the proximal end of the catheter. The drive unit includes at least one rotatable transformer and a motor. The at least one rotatable transformer includes a rotor and a stator. The rotor is coupled to the proximal end of the driveshaft. The motor is for driving rotation of the driveshaft.

In yet another embodiment, a method for photo-acoustic imaging of a patient using an intravascular ultrasound imaging system includes inserting a catheter into patient vasculature. The catheter includes at least one rotatable light source coupled to a control module, and at least one rotatable transducer electrically coupled to a control module. The at least one light source rotates with the at least one transducer and maintains a constant position and direction relative to the at least one transducer. Patient tissue is illuminated with light emitted from the light source. At least one emitted acoustic signal is received from the illuminated patient tissue. At least one acoustic signal is transmitted to patient tissue from at least one transducer. At least one reflected acoustic signal is received from the patient tissue.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an intravascular ultrasound imaging system, according to the invention;

FIG. 2 is a schematic side view of one embodiment of a catheter of an intravascular ultrasound imaging system, according to the invention;

FIG. 3 is a schematic perspective view of one embodiment of a distal end of the catheter shown in FIG. 2 with an imaging core disposed in a lumen defined in the catheter, according to the invention;

FIG. 4A is a schematic perspective view of one embodiment of a distal end of a catheter for an IVUS imaging system, the catheter including a light source and at least one transducer, according to the invention;

FIG. 4B is a schematic longitudinal cross-sectional view of the distal end of the catheter shown in FIG. 4A, the catheter including a light source and at least one transducer, according to the invention;

FIG. 4C is a schematic longitudinal cross-sectional view of the distal end of the catheter shown in FIG. 4A, the catheter including a light source and at least one transducer, the catheter also including a light director for directing light emitted from the light source, according to the invention; and

FIG. 5 is a schematic cross-sectional view of one embodiment of a proximal end of the catheter shown in FIG. 4A coupled to a drive unit, according to the invention;

### DETAILED DESCRIPTION

The present invention is directed to the area of intravascular ultrasound imaging systems and methods of making and using the systems. The present invention is also directed to intravascular ultrasound systems that also include photo-acoustic imaging, as well as methods of making and using the intravascular ultrasound systems.

Suitable intravascular ultrasound (“IVUS”) imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,306,561; and 6,945,938; as well as U.S. Patent Application Publication Nos. 20060253028; 20070016054; 20070038111; 20060173350; and 20060100522, all of which are incorporated by reference.

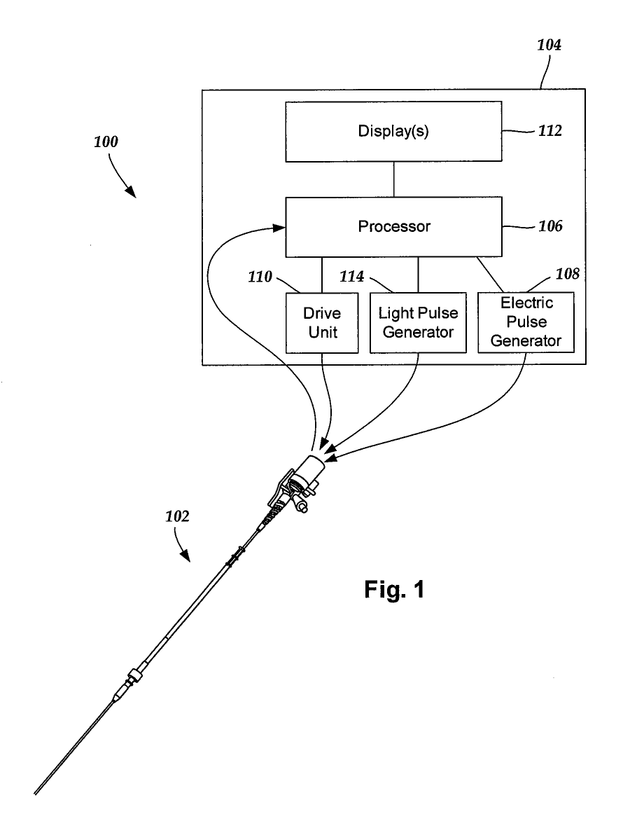

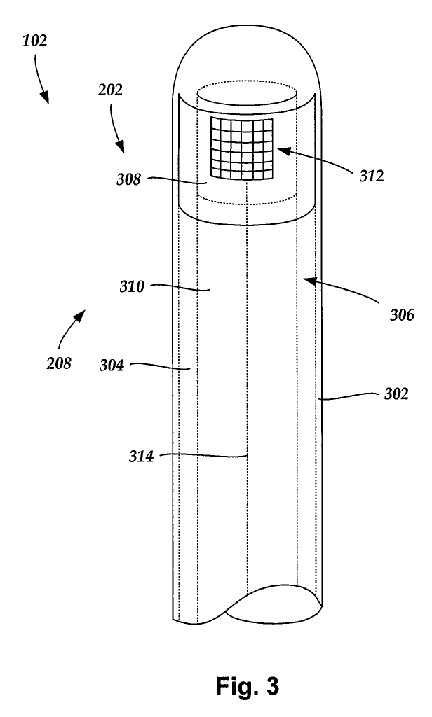

FIG. 1 illustrates schematically one embodiment of an IVUS imaging system 100. The IVUS imaging system 100 includes a catheter 102 that is coupleable to a control module 104. The control module 104 may include, for example, a processor 106, an electric pulse generator 108, a drive unit 110, and one or more displays 112. In at least some embodiments, the electric pulse generator 108 forms electric pulses that may be input to one or more transducers (312 in FIG. 3) disposed in the catheter 102. In at least some embodiments, mechanical energy from a motor disposed within the drive unit 110 may be used to drive an imaging core (306 in FIG. 3) disposed in the catheter 102. In at least some embodiments, the drive unit 110 additionally includes a transformer.

In at least some embodiments, electric pulses transmitted from the one or more transducers (312 in FIG. 3) may be input to the processor 106 for processing. In at least some embodiments, the processed electric pulses from the one or more transducers (312 in FIG. 3) may be displayed as one or more images on the one or more displays 112. In at least some embodiments, the processor 106 may also be used to control the functioning of one or more of the other components of the control module 104. For example, the processor 106 may be used to control at least one of the frequency or duration of the electrical pulses transmitted from the electric pulse generator 108, the rotation rate of the imaging core (306 in FIG. 3) by the drive unit 110, the velocity or length of the pullback of the imaging core (306 in FIG. 3) by the drive unit 110, or one or more properties of one or more images formed on the one or more displays 112. A light pulse generator 114 is provided to generate electric signals that direct a light source at a distal end of the catheter 102 to generate light to illuminate patient tissue in proximity to the catheter 102, as discussed in more detail below.

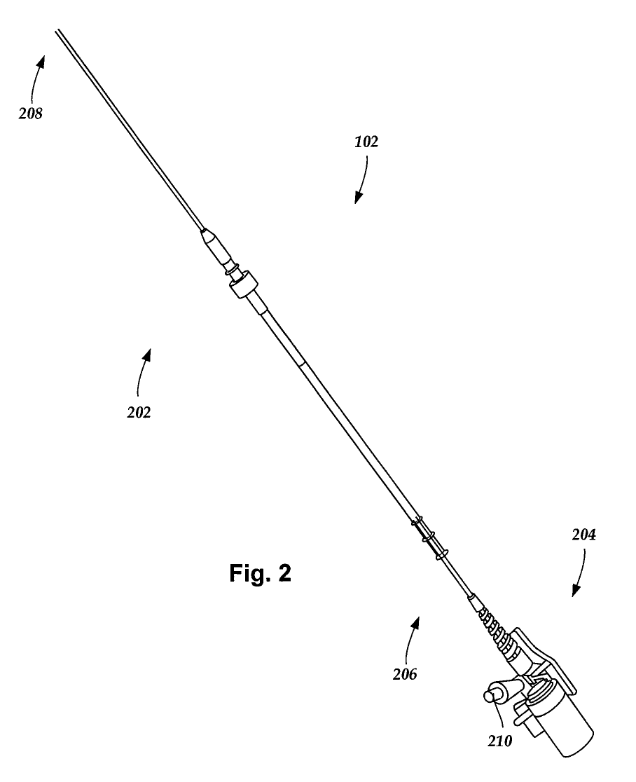

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. In at least some embodiments, the catheter 102 defines at least one flush port, such as flush port 210. In at least some embodiments, the flush port 210 is defined in the hub 204. In at least some embodiments, the hub 204 is configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the elongated member 202 of the catheter 102. The elongated member 202 includes a sheath 302 and a lumen 304. An imaging core 306 is disposed in the lumen 304. The imaging core 306 includes an imaging device 308 coupled to a distal end of a rotatable driveshaft 310.

The sheath 302 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

One or more transducers 312 may be mounted to the imaging device 308 and employed to transmit and receive acoustic pulses. In a preferred embodiment (as shown in FIG. 3), an array of transducers 312 are mounted to the imaging device 308. In other embodiments, a single transducer may be employed. In yet other embodiments, multiple transducers in an irregular-array may be employed. Any number of transducers 312 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used.

The one or more transducers 312 may be formed from one or more known materials or devices capable of transforming applied electrical pulses to pressure distortions on the surface of the one or more transducers 312, and vice versa. Examples of suitable materials or devices include piezoelectric ceramic materials, piezocomposite materials, piezoelectric plastics, barium titanates, lead zirconate titanates, lead metaniobates, polyvinylidenefluorides, capacitive micromachined ultrasonic transducers, and the like.

The pressure distortions on the surface of the one or more transducers 312 form acoustic pulses of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic pulses of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 312 may include a layer of piezoelectric material sandwiched between a conductive acoustic lens and a conductive backing material formed from an acoustically absorbent material (e.g., an epoxy substrate with tungsten particles). During operation, the piezoelectric layer may be electrically excited by both the backing material and the acoustic lens to cause the emission of acoustic pulses.

In at least some embodiments, the one or more transducers 312 can be used to form a radial cross-sectional image of a surrounding space. Thus, for example, when the one or more transducers 312 are disposed in the catheter 102 and inserted into a blood vessel of a patient, the one more transducers 312 may be used to form an image of the walls of the blood vessel and tissue surrounding the blood vessel.

The imaging core 306 is rotated about a longitudinal axis of the catheter 102. As the imaging core 306 rotates, the one or more transducers 312 emit acoustic pulses in different radial directions. When an emitted acoustic pulse with sufficient energy encounters one or more medium boundaries, such as one or more tissue boundaries, a portion of the emitted acoustic pulse is reflected back to the emitting transducer as an echo pulse. Each echo pulse that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to form a displayable image of the imaged region based, at least in part, on a collection of information from each of the acoustic pulses transmitted and the echo pulses received.

As the one or more transducers 312 rotate about the longitudinal axis of the catheter 102 emitting acoustic pulses, a plurality of images are formed that collectively form a radial cross-sectional image of a portion of the region surrounding the one or more transducers 312, such as the walls of a blood vessel of interest and the tissue surrounding the blood vessel. In at least some embodiments, the radial cross-sectional image can be displayed on one or more displays 112.

In at least some embodiments, the imaging core 306 may move longitudinally within the lumen of the catheter 102 while the catheter 102 remains stationary. For example, the imaging core 306 may be advanced (moved towards the distal end of the catheter 102) or retracted/pulled back (moved towards the proximal end of the catheter 102) within the lumen 304 of the catheter 102 while the catheter 102 remains in a fixed location within patient vasculature (e.g., blood vessels, the heart, and the like). During longitudinal movement (e.g., pullback) of the imaging core 306, an imaging procedure may be performed, wherein a plurality of cross-sectional images are formed along a longitudinal length of patient vasculature.

In at least some embodiments, the catheter 102 includes at least one retractable section that can be retracted during an imaging procedure. In at least some embodiments, a motor disposed in the drive unit (110 in FIG. 1) drives the pullback of the imaging core 306 within the catheter 102. In at least some embodiments, the pullback distance of the imaging core is at least 5 cm. In at least some embodiments, the pullback distance of the imaging core is at least 10 cm. In at least some embodiments, the pullback distance of the imaging core is at least 15 cm. In at least some embodiments, the pullback distance of the imaging core is at least 20 cm. In at least some embodiments, the pullback distance of the imaging core is at least 25 cm.

The quality of an image produced at different depths from the one or more transducers 312 may be affected by one or more factors including, for example, bandwidth, transducer focus, beam pattern, as well as the frequency of the acoustic pulse. The frequency of the acoustic pulse output from the one or more transducers 312 may also affect the penetration depth of the acoustic pulse output from the one or more transducers 312. In general, as the frequency of an acoustic pulse is lowered, the depth of the penetration of the acoustic pulse within patient tissue increases. In at least some embodiments, the IVUS imaging system 100 operates within a frequency range of 5 MHz to 60 MHz.

In at least some embodiments, one or more conductors 314 electrically couple the transducers 312 to the control module (104 in FIG. 1). In at least some embodiments, the one or more conductors 314 extend along a longitudinal length of the imaging core 306. In at least some embodiments, the one or more conductors 314 may extend along at least a portion of the longitudinal length of the catheter 102 as shielded electrical cables, such as a coaxial cable, or a twisted pair cable, or the like.

In at least some embodiments, the catheter 102 with one or more transducers 312 mounted to the distal end 208 of the imaging core 306 may be inserted percutaneously into a patient via an accessible blood vessel, such as the femoral artery, at a site remote from the selected portion of the selected region, such as a blood vessel, to be imaged. The catheter 102 may then be advanced through the blood vessels of the patient to the selected imaging site, such as a portion of a selected blood vessel.

Differentiating between two or more different tissue types displayed on an IVUS image is desirable, but can be difficult using the IVUS image. For example, it may be difficult to determine where a border between two or more tissue types is located, or even if a border exists.

One technique for tissue differentiation is photo-acoustic imaging, wherein patient tissue is pulsed with light from a light source, such as a laser diode. When patient tissue is pulsed with light, some of the emitted light is absorbed by the tissue and converted to heat. The heat causes a transient ultrasonic expansion of the illuminated tissue and a corresponding ultrasonic emission, which may be received by one or more transducers and processed into a displayable image.

Photo-acoustic imaging capabilities may be incorporated into an IVUS imaging system. Such an arrangement includes one or more transducers disposed at a distal end of catheter and a light source also disposed at the distal end of the catheter in proximity to the one or more transducers. Light emitted from the light source is directed to patient tissue such that the subsequently-emitted acoustic pulses from the illuminated tissue may be received by the one or more transducers. It is desirable to have the light source and the one or more transducers both be disposed on the imaging core within the catheter so that the light source rotates with the one or more transducers, thereby maintaining a constant relative position with respect to the one or more transducers.

Previous systems have embedded optical fibers in a sheath of a catheter. However, embedding optical fibers in the sheath can make sheath manufacturing difficult. Moreover, the embedded optical fibers do not rotate with transducers. Additionally, embedding optical fibers in the sheath may hinder, or even eliminate, the pullback function of the imaging core during an imaging procedure.

In at least some embodiments, an IVUS imaging system incorporates photo-acoustic imaging capabilities into the IVUS imaging system. One or more light sources (e.g., laser diodes, or the like) are disposed in an imaging core of the IVUS imaging system. In at least some embodiments, the one or more light sources disposed in the imaging core couple to the distal end(s) of one or more conductors extending along a longitudinal length of the imaging core. In at least some embodiments, the proximal end(s) of the one or more conductors are coupled to a transformer disposed in a drive unit of the IVUS imaging system.

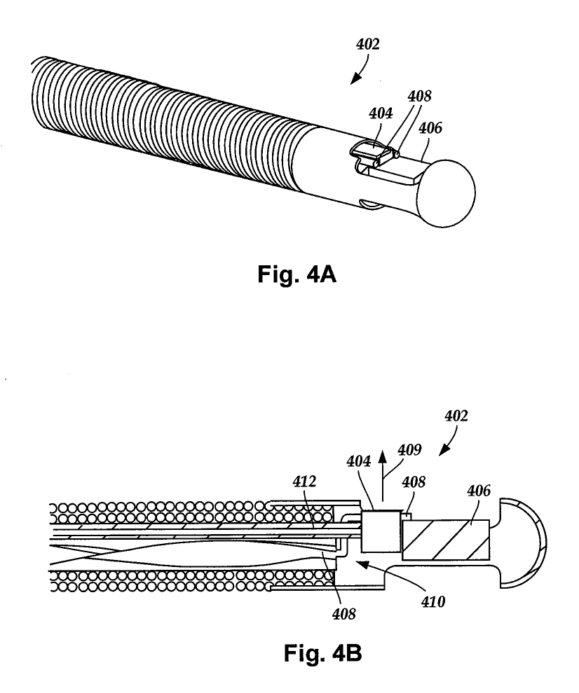

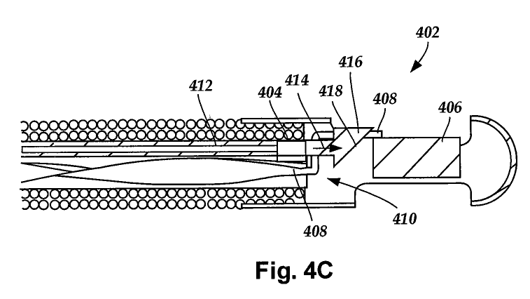

FIG. 4A is a schematic perspective view of one embodiment of a distal end of a catheter 402 for an IVUS imaging system (100 in FIG. 1). The catheter 402 includes a light source 404 (e.g., a laser diode, or the like) and one or more transducers 406. The one or more transducers 406 are coupled to the processor (106 in FIG. 1) via one or more electrical conductors 408 disposed in an imaging core (410 in FIG. 4B). In at least some embodiments, the one or more electrical conductors 408 provide power to the one or more transducers 406. In at least some embodiments, the one or more electrical conductors 408 provide signals to and from the transducers 406.

In at least some embodiments, the light source 404 is configured and arranged such that light emitted from the light source 404 is directed outward from the catheter 402, as shown by directional arrow 409. In at least some embodiments, the light source 404 is configured and arranged such that light emitted from the light source 404 is directed outward from the catheter 402 in a direction that is approximately perpendicular to a longitudinal axis of the distal end of the catheter 402. In at least some embodiments, a diffuser (see e.g., 416 in FIG. 4C) is positioned over the light source 404 to diffuse light emitted from the light source 404.

FIG. 4B is a schematic longitudinal cross-sectional view of one embodiment of a distal end of the catheter 402. The catheter 402 includes a lumen into which an imaging core 410 is disposed. In at least some embodiments, the imaging core 410 includes the one or more electrical conductors 408 extending along at least a portion of the imaging core 410. The light source 404 is coupled to the processor (106 in FIG. 1) via one or more electrical conductors 412 disposed in an imaging core (410 in FIG. 4B). In at least some embodiments, the one or more electrical conductors 412 provide power to the light source 404. In at least some embodiments, the one or more electrical conductors 412 provide electrical signals to and from the light source 404.

In at least some embodiments, the one or more electrical conductors 408 may extend along at least a portion of the longitudinal length of the catheter 402 as shielded electrical cables, such as a coaxial cable, or a twisted pair cable, or the like. In at least some embodiments, the one or more electrical conductors 412 may extend along at least a portion of the longitudinal length of the catheter 402 as shielded electrical cables, such as a coaxial cable, or a twisted pair cable, or the like. In at least some embodiments, the one or more electrical conductors 412 wrap at least one time around the one or more electrical conductors 408. In at least some embodiments, the light source 404 and the one or more transducers 406 both use the same one or more conductors.

In at least some embodiments, the light source 404 and the one or more transducers 406 have the same rotational velocity. In at least some embodiments, the light source 404 maintains a constant relative position with respect to the one or more transducers 406. In at least some embodiments, the light source 404 is fixed to the one or more transducers 406. In some embodiments, the light source 404 is proximal to the one or more transducers 406. In other embodiments, the light source 404 is distal to the one or more transducers 406.

Light provided from a light source 404 may be used to illuminate selected patient tissue for photo-acoustic imaging. In at least some embodiments, the light may be emitted in one or more timed patterns, such as pulses.

In at least some embodiments, the IVUS imaging system may be used to perform photo-acoustic imaging without performing ultrasound imaging. In at least some embodiments, the IVUS imaging system is configured to perform both photo-acoustic imaging and ultrasound imaging, either sequentially or independently. In at least some embodiment, the data from a photo-acoustic image and an ultrasound image may be combined to form a composite image.

FIG. 4C is a schematic longitudinal cross-sectional view of another embodiment of a distal end of the catheter 402. The light source 404 is configured and arranged such that light emitted from the light source 404 is directed along the longitudinal length of the distal end of the catheter 402, as indicated by directional arrow 414, and redirected by a light director 416. In at least some embodiments, the light director 416 includes a mirror 418 to redirect the light from the light source 404 to a desired tissue. In at least some embodiments, the light director 416 includes a diffuser to diffuse light from a narrow point source (e.g., a laser diode, or the like). In at least some embodiments, the light director 416 includes a mirror 418 and a diffuser. In at least some embodiments, the mirror 418 and the diffuser are separate from one another. In at least some embodiments, the mirror 418 has a light-diffusing reflective surface. The light director 416 may be fabricated from any material suitable for reflecting or orienting light including, for example, glass, plastic, and the like or combinations thereof.

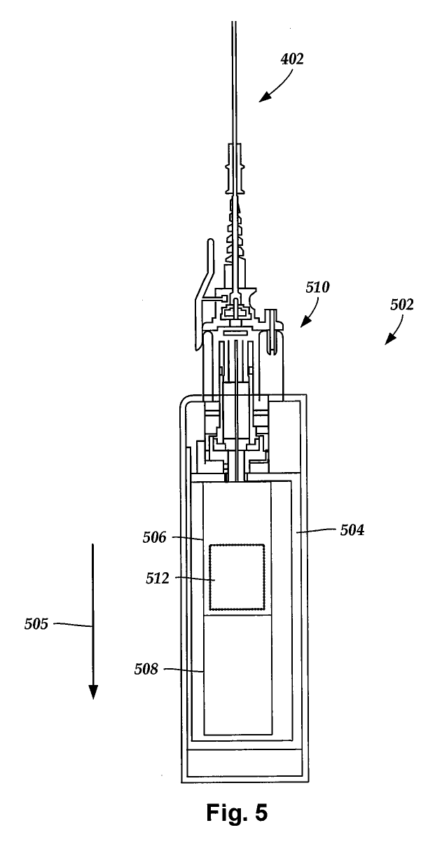

In at least some embodiments, a proximal end of the catheter 402 couples with at least one transformer disposed in a drive unit. FIG. 5 is a schematic cross-sectional view of one embodiment of a proximal end of the catheter 402 coupled to a drive unit 502. The drive unit 502 includes a drive sled 504 configured and arranged to slide along a length of the drive unit 502 during pullback of the imaging core (410 in FIG. 4B) in the direction shown by directional arrow 505, thereby retracting the proximal end of the catheter 402. The drive unit 502 also includes a transformer 506 and a motor 508. In FIG. 5, the transformer 506 and the motor 508 are shown coupled to the drive sled 504.

In at least some embodiments, the motor 508 drives the rotation of the imaging core (410 in FIG. 4B) and a rotating portion of the transformer 506. The transformer 506 is coupled to the one or more electrical conductors 408 and also to the control module (104 in FIG. 1) and allows signals to pass between the stationary control module (104 in FIG. 1) and the rotating imaging core (410 in FIG. 4B). In at least some embodiments, the transformer 506 is also coupled to the one or more electrical conductors 412. In at least some other embodiments, the one or more electrical conductors 412 are coupled to another transformer 512. In at least some embodiments, when the one or more electrical conductors 412 are coupled to the transformer 512, the transformer 512 is disposed inside the transformer 506.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A catheter assembly for an intravascular ultrasound system, the catheter assembly comprising:
a catheter having a distal end, a proximal end, and a longitudinal length, the catheter defining a lumen extending along the longitudinal length of the catheter from the proximal end to the distal end; and
an imaging core configured and arranged for inserting into the lumen, the imaging core comprising
a rotatable driveshaft having a distal end, a proximal end, and a longitudinal length,
at least one light source disposed at the distal end of the rotatable driveshaft, the at least one light source configured and arranged for rotating with the driveshaft and also for transforming applied electrical signals to light for illuminating an object in proximity to the catheter, and
at least one transducer disposed at the distal end of the rotatable driveshaft, the at least one transducer configured and arranged for rotating with the driveshaft, wherein the at least one transducer is configured and arranged for receiving acoustic signals generated by the object in response to illumination of the object by the light emitted from the at least one light source.

2. The catheter assembly of claim 1, wherein the imaging core further comprises at least one light director configured and arranged for directing light emitted from the at least one light source towards a desired tissue region.

3. The catheter assembly of claim 2, wherein the at least one light director comprises a mirror.

4. The catheter assembly of claim 2, wherein the at least one light director comprises a diffuser.

5. The catheter assembly of claim 1, wherein the at least one light source is a laser diode.

6. The catheter assembly of claim 1, wherein the at least one transducer is configured and arranged for transforming applied electrical signals to acoustic signals, and also for transforming received acoustic signals to electrical signals.

7. The catheter assembly of claim 1, wherein the at least one light source is disposed on the imaging core proximal to the at least one transformer.

8. The catheter assembly of claim 6, wherein the at least one light source is disposed on the imaging core distal to the at least one transformer.

9. The catheter assembly of claim 1, wherein the at least one light source is configured and arranged to generate pulses of light at one or more selected frequencies.

10. An intravascular ultrasound imaging system comprising:
a catheter having a distal end, a proximal end, and a longitudinal length, the catheter defining a lumen extending along the longitudinal length of the catheter from the proximal end to the distal end;
an imaging core configured and arranged for inserting into the lumen, the imaging core comprising
a rotatable driveshaft having a distal end, a proximal end, and a longitudinal length,
at least one light source disposed at the distal end of the rotatable driveshaft, the at least one light source configured and arranged for rotating with the driveshaft and also for transforming applied electrical signals to light for illuminating an object in proximity to the catheter, and
at least one transducer disposed at the distal end of the rotatable driveshaft, the at least one transducer configured and arranged for rotating with the driveshaft, wherein the at least one transducer is configured and arranged to receive acoustic signals generated by the object in response to illumination of the object by the light emitted from the light source; and
a drive unit coupled to the proximal end of the catheter, the drive unit comprising
at least one rotatable transformer comprising a rotor and a stator, wherein the rotor is coupled to the proximal end of the driveshaft, and
a motor for driving rotation of the driveshaft.

11. The system of claim 10, further comprising a control module coupled to the imaging core, the control module comprising
an electric pulse generator configured and arranged for providing electric signals to the at least one transducer, the electric pulse generator electrically coupled to the at least one transducer via the one or more electrical conductors and at least one of the rotatable transformers;
a light source controller configured and arranged for providing electric signals to the at least one light source, the light source controller electrically coupled to the at least one light source via the one or more electrical conductors and at least one of the rotatable transformers; and
a processor configured and arranged for processing received electrical signals from the at least one transducer to form at least one image, the processor electrically coupled to the at least one transducer via the one or more electrical conductors and at least one of the rotatable transformers.

12. The system of claim 11, wherein the at least one transducer and the at least one light source are each coupled to a different one of the at least one rotatable transformers.

13. The system of claim 11, wherein the at least one transducer and the at least one light source are each coupled to the same one of the at least one rotatable transformers.

14. The system of claim 11, wherein the control module further comprises at least one display electrically coupled to the processor, the at least one display configured and arranged for displaying the at least one image formed by the processor.

15. The system of claim 11, wherein the at least one light source is configured and arranged to generate pulses of light.

16. The system of claim 11, wherein the at least one light source is a laser diode.

17. A method for photo-acoustic imaging of a patient using an intravascular ultrasound imaging system, the method comprising:
inserting a catheter into patient vasculature, the catheter comprising at least one rotatable light source coupled to a control module and at least one rotatable transducer electrically coupled to a control module, wherein the at least one light source rotates with the at least one transducer and maintains a constant position and direction relative to the at least one transducer;
illuminating patient tissue with light emitted from the light source;
receiving at least one emitted acoustic signal from the illuminated patient tissue;
transmitting at least one acoustic signal to patient tissue from the at least one transducer; and
receiving at least one reflected acoustic signal from the patient tissue.

18. The method of claim 17, wherein the received at least one emitted acoustic signal and the received at least one reflected acoustic signal are transmitted to a processor for processing.

19. The method of claim 18, further comprising displaying an image based on the received and processed at least one emitted acoustic signal and the received and processed at least one reflected acoustic signal.

20. The method of claim 17, wherein illuminating patient tissue with light emitted from the light source comprises passing the emitted light from the light source through a light director.
